(19) United States
(12) Patent Application Publication
(10) Pub. No.: US 2021/0268183 A1
(43) Pub. Date: Sep. 2, 2021

(54) METHOD OF CONTROLLING FLUID FLOW EXITING INFUSION PUMPS

(71) Applicants: (lNSERM) INSTITUT NATIONAL DE LA SANTÉ ET DE LA RECHERCHE MÉDICALE, Paris (FR); UNIVERSITÉ PARIS-SACLAY, Orsay (FR); THE UNIVERSITY OF WARWICK, Warwickshire (GB)

(72) Inventors: Annabelle BALLESTA, Villejuif (FR); Roger HILL, Warwickshire (GB); Francis LEVI, Warwickshire (GB)

(21) Appl. No.: 17/257,666

(22) PCT Filed: Jul. 2, 2019
(86) PCT No.: PCT/EP2019/067691

§ 371 Date: Jan. 4, 2021

(30) Foreign Application Priority Data
Jul. 3, 2018 (EP) 18181320.5

(51) Int. Cl. A61M 5/168; A61M 5/142; G16H 20/17

(52) U.S. Cl. CPC A61M 5/16877; A61M 2205/3334; G16H 20/17; A61M 5/142

(57) ABSTRACT

The present invention relates to a method of controlling the flow of a fluid in a tube (11) of an infusion pump (1), in particular for chronomodulated administration of anticancer drugs. The inventors found that the actual drug flow exiting such a pump has unexpected time delays and/or drug delivery spikes. The proposed method comprises an estimation of the temporal evolution of the concentration of drug or other substances at an exit end (E11) of said tube (11) as a function of a size of said tube (11). Typically, the size of the tube (11) may be its length (L11) and said concentration may be estimated according to a transport equation.

The present invention relates generally to infusion pumps, in particular those used for chronomodulated administration of anticancer drugs.

In accordance with the present invention, there is provided a method of controlling the flow of a fluid exiting an infusion pump, and corresponding infusion pump, computer program and computer-readable medium.

Infusion pumps are chronotherapeutic systems designed to deliver a plurality of drug substances, typically up to four drug substances, according to pre-programmed schedules over the circadian (24-h) time period.

A typical infusion pump and applications are for example described in the following document: M. H. Smolensky and N. A. Peppas. Chronobiology, drug delivery, and chronotherapeutics. Advanced Drug Delivery Reviews, 59(9-10):828-851, 2007.

The infusion pump can comprise four drug bags each with a storage capacity of 100 mL. Three of those bags may be filled with a drug substance such as Irinotecan, 5-Fluorouracil or Oxaliplatin. The fourth bag may be filled with a glucose serum. The drug bags are connected to a distribution manifold arranged to inject into a distributing tube a fluid that comprises either a selected combination of the drug substances or the glucose serum.

In a simplified illustrative example, an injection cycle may comprise:
a first stage in which one drug substance is delivered as a 6-h sinusoidal infusion,
a second stage consisting in a glucose serum flush to rinse the tubing,
a third stage in which another drug substance is delivered as a 6-h sinusoidal infusion.

Drug substance administration using this type of infusion pump being now a long-established practice, the inventors were surprised to find that the actual drug flow exiting the pump has unexpected time delays and/or drug delivery spikes.

It is an object of the present invention to optimize the functioning of an infusion pump and to provide a method that is capable of controlling the fluid flow exiting the infusion pump without the drawbacks associated with the prior art.

In that respect, the present invention relates to a method of controlling the flow of a fluid in a tube of an infusion pump. The fluid comprises at least one substance. This method comprises an estimation of the temporal evolution of the concentration of said at least one substance at an exit end of said tube as a function of a size of said tube.

In a preferred embodiment, said size of the tube may be the length or the inner diameter or the inner volume of the tube.

Using this method, the fluid flow actually exiting the pump can thus be controlled taking into account the structure of the pump and more specifically of the tube. Time delays and unwanted drug delivery spikes can therefore be avoided.

According to an aspect of the invention, the method may comprise injecting the fluid into the tube according to a predetermined temporal injection profile, said estimation being a function of this injection profile.

In particular, said temporal evolution of the concentration of the at least one substance along the tube may be estimated according to the following equation:

$$\frac{\partial U}{\partial r} = - V ( t ) \frac{\partial U}{\partial x} + D \frac{{\partial }^{2} U}{\partial {x}^{2}} + \partial ( x ) S ( t ) x ∈ [ 0 , L 11 ]$$

where U is the concentration of the at least one substance, V(t) is the velocity of the fluid flowing along the tube, D is a predetermined diffusion coefficient of the at least one substance, x is the coordinate along the tube from its inlet end where x=0 to its exit end where x=L11, L11 is the length of the tube, t is the time, ∂(x) is an indicator function which is equal to 1 at x=0 and 0 otherwise, and S(t) is the predetermined temporal injection profile.

This transport equation provides a relevant estimate of the fluid flow exiting the pump tube so that an infusion pump programmed accordingly would be particularly suitable for chronotherapeutic applications.

The following initial condition is preferably added where ƒ⁰ is a function describing the substance quantity in the tube at the initial time: U(0,x)=ƒ⁰(x).

In an embodiment, said injection profile may be sinusoidal.

In another embodiment, temporal evolution of the concentration of the at least one substance along the tube may be estimated according to the following equations:

$$\{ \begin{matrix}\frac{\partial u ( x , t )}{\partial t} + V ( t ) \frac{\partial u ( x , t )}{\partial x} = 0 , \\ s a * V ( t ) u ( 0 , t ) = S ( t )\end{matrix}$$

where the constant sa=pi*r² is the cross sectional surface area of the tube (in m²), with r being the radius of the tube. The source term S(t) representing the infusion profile programmed into the pump, is expressed in mol/h. Initial conditions along the tube are u(x,0)=0. The fluid velocity and source terms are controlled by the pump which imposes a fluid delivery rate expressed in ml/h. They are defined by converting the fluid delivery rate into mol/h and mm/h respectively using the tube geometry and the concentration of each drug solution. Hence, model simulations at the end of the tube (x=L11) do not depend on the exact geometry of the tube but rather on its total volume.

The transport equation with associated initial and boundary conditions can be solved using the classical method of characteristics which gives:

$$u ( t , x ) = \{ \begin{matrix}0 if {\int }_{0}^{c} V ( r ) d r < x \\ \frac{S ( τ ( t , x ) )}{s a V ( τ ( t , x ) )} otherwise with {\int }_{τ ( t , x )}^{c} V ( r ) d r = x\end{matrix}$$

Hence, model simulations at the end of the tube give the rate of drug infusion into the patient (i.e. at x=L11) and can be obtained by:

$$d ( t ) = s a * V ( T ) * u ( t , L 11 ) = \{ \begin{matrix}0 & for t such that {\int }_{0}^{t} V ( r ) d r < L 11 \\ V ( t ) \frac{S ( {τ}_{L} ( t ) )}{V ( {τ}_{L} ( t ) )} & otherwise , with L 11 = {\int }_{{τ}_{L} ( t )}^{c} V ( r ) d r\end{matrix}$$

Note that, for infusion of solutions containing a drug, the source term S(t) is proportional to the fluid velocity V(t) as the drug is infused within the tube in the same time as the fluid, so that d(t) is proportional to V(t) once the tube is filled i.e. for times t such that ∫₀^{t}V(r)dr>L11.

Based on this new formulation, the rate of drug infusion into the patient can be computed more efficiently and with better precision.

Others injection profiles may be used as described below.

According to an aspect of the invention, the method may comprise at least one injection cycle comprising in sequence:
a first stage in which a fluid is injected into the tube so as to fill to its exit end with said fluid,
a second stage in which a fluid is injected into the tube according to a first predetermined temporal injection profile,
a third consecutive stage in which a fluid is injected into the tube according to a second predetermined temporal injection profile.

Two consecutive temporal injection profiles may be designed to avoid undesired spikes, i.e. concentration of substances exiting the tube at undesired time. For example, the fluid injected in the tube may comprise a drug substance during the second stage and only glucose serum during the third stage so that the drug substance residue within the tube at the end of the second stage can be delivered during the third stage according to the desired distribution profile. In other words, the second and third stages can be designed so that both first and second injection profiles allow distribution of drug substances according to a unique temporal injection profile, despite the inevitable presence of drug substance residue within the tube at the end of the second stage.

In an embodiment, during the first stage, the fluid can be injected into the tube according to an injection profile defined by the following equation: S0(t)=r₀, where r₀ is a coefficient having a predetermined value determined so that the tube is filled to its exit end with the fluid at the end of the first stage.

In a particular embodiment, the first injection profile can be defined by the following equation: S1(t)=f(t) with T₁<t<T₂, T₁ being the time at which the second stage starts and T₂ the time at which the second stage ends, and where f(t) is a function, for example a continuous function. The second injection profile can be defined by the following equation: S2(t)=f(t) with T₂<t<T_{end}, T₂ being the time at which the third stage starts and T_{end} the time at which the third stage ends.

T₂ can be calculated by Ω=V_{tube}+∫_{T}_{₁}^{T}^{₂}ƒ(t)dt, where V_{tube} is the inner volume of the tube and Ω the total volume of drug solution to be delivered.

The invention also relates to an infusion pump having a tube configured to flow a fluid therethrough and means adapted to carry out the method described above.

In other forms, the invention also relates to a computer program comprising instructions to cause the infusion pump to carry out this method, and to a computer-readable medium having stored thereon this computer program.

The above and others features, details and advantages of the present invention will become apparent from the following description and the accompanying drawings in which:

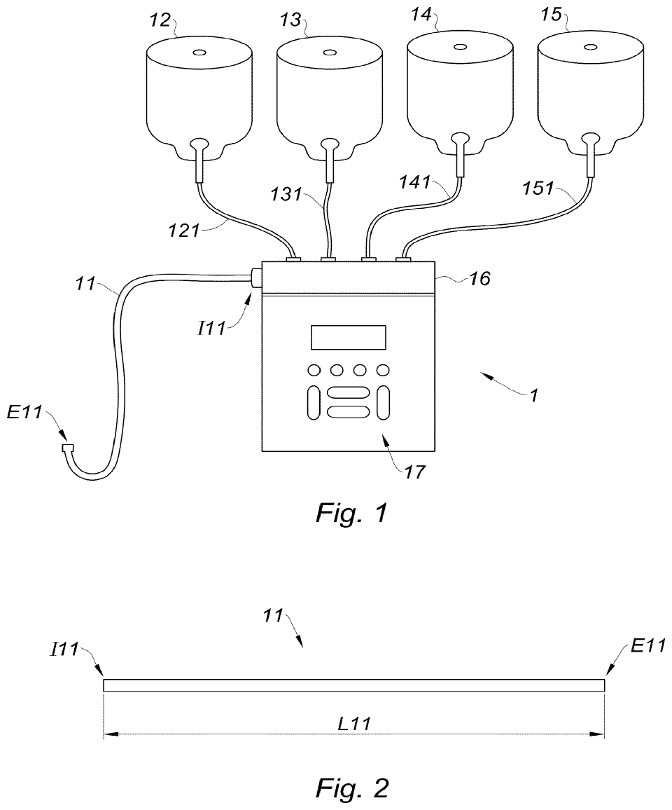

FIG. 1 is a schematic view of an infusion pump according to the invention;

FIG. 2 is a schematic view of a tube of the infusion pump of FIG. 1;

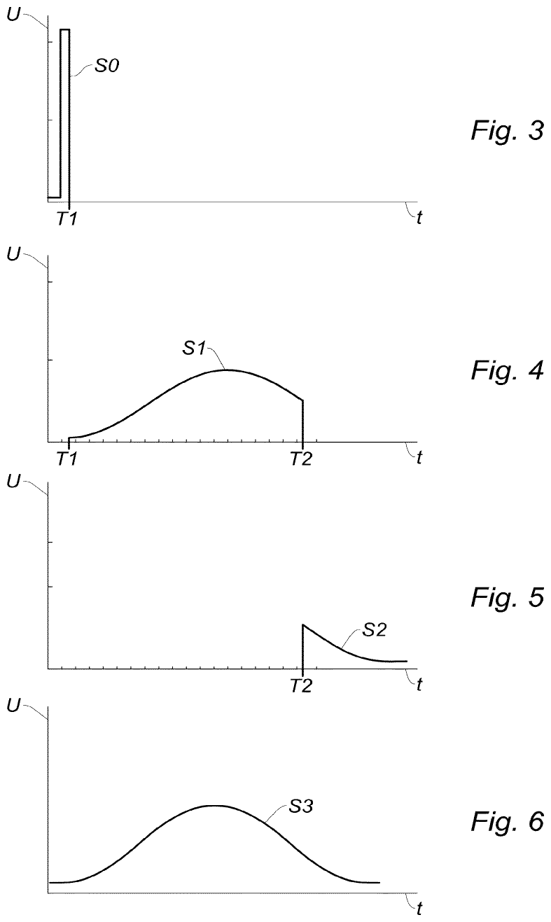

FIGS. 3 to 5 illustrate an injection cycle according to the invention;

FIG. 6 shows a sinusoidal infusion profile.

Identical or similar elements are marked with identical reference signs in all of the figures.

An embodiment of the infusion pump of the invention is shown in FIG. 1.

This infusion pump 1 comprises four bags 12-15. In this example, the bag 12 is filled with a first substance such as glucose serum, and the bags 12-14 are respectively filled with three different drug substances, for example Irinotecan, 5-Fluorouracil and Oxaliplatin.

The pump 1 has a distribution manifold 16. The bags 12-15 are respectively connected to this manifold 16 so that a control unit (not represented) of the pump 1 can control the amount of each type of substance arriving within the manifold 16 via corresponding pipes 121-151.

This arrangement results in that the distribution manifold 16 can thus be filled with a fluid that comprise at least one substance from at least one of the bag, in particular either a combination of said drug substances or the glucose serum.

The distribution manifold 16 is arranged to inject this fluid into a tube 11 of the pump 1 to extract the fluid from the pump 1.

As illustrated in FIGS. 1 and 2, the tube 11 has an inlet E11 connected to the distribution manifold 16, and an exit end E11 from which the fluid can be extracted from the pump 1.

In an embodiment, the fluid is injected into the tube 11 according to a predetermined temporal injection profile such as the sinusoidal profile S3 illustrated in FIG. 6.

To control the fluid flow in the tube 11, the control unit of the pump 1 calculates an estimation of the temporal evolution of the concentration of said at least one substance at the exit end E11 of the tube 11, in this example as a function of the length L11 of the tube 11.

In that respect, the pump 1 may comprise a keypad 17 allowing a user to select or define the length and/or any other size of the tube 11 such as the inner diameter of the tube 11 or its inner volume.

It is preferred to calculate said estimation also as a function of said injection profile, whether this profile is sinusoidal (e.g. profile S3) or of any other type.

The inventors found that the following equation provides a good estimation of the fluid flow along the tube 11:

$$\frac{\partial U}{\partial t} = - V ( t ) \frac{\partial U}{\partial x} + D \frac{{\partial }^{2} U}{\partial {x}^{2}} + \partial ( x ) S ( t ) x ∈ [ 0 , L 11 ]$$

where U is the concentration of the at least one substance, V(t) is the velocity of the fluid flowing along the tube 11, D is a predetermined diffusion coefficient, x is the coordinate along the tube 11 from its inlet 111 end where x=0 to its exit end E11 where x=L11, L11 is the length of the tube, t is the time, ∂(x) is an indicator function which is equal to 1 at x=0 and 0 otherwise, and S(t) is the predetermined temporal injection profile.

The diffusion coefficient can be chosen in the range from 10⁻⁶ to 10 mm/h, knowing that the person skilled in the art can easily determine the exact values to be applied using conventional experimental procedures.

Of course, the parameters and/or functions of this equation can be modified from a stage to another stage of an injection cycle. In particular, it is considered to use different profiles S(t) at different stages depending on the substance or combination of substances to be injected during those different stages.

In a specific embodiment, an injection cycle comprises the three following stages.

In a first stage, a fluid comprising for example a given combination of substances is injected into the tube so as to fill to its exit end with this fluid. FIG. 3 shows a temporal injection profile S0 that could be implemented during this first stage. In this example, the tube 11 is completely filled at time T1.

In a second stage extending over period T1 to T2, the same fluid can be injected into the tube according to the temporal injection profile S1 illustrated in FIG. 4. The profile S1 is here a first section of a sinusoidal profile: the injection rate of said combination of substances starts raising at T1 so that, when implemented in the above transport equation, the fluid flow actually begins exiting the tube 11 because the tube is completely filled at T1 (see first stage above).

T2 is defined as the time at which all drug has entered the beginning of tube 11. This can be calculated by Ω=V_{tube}+∫_{T}_{₁}^{T}^{₂}ƒ(t)dt, where ƒ is the injection profile, V_{tube} the inner volume of the tube and Ω the total volume of drug solution to be delivered.

In a third stage extending from T2, a fluid comprising another substance such as a glucose serum is injected into the tube 11 according to the temporal injection profile S2 illustrated in FIG. 5. The profile S2 is here a second section of the injection function ƒ(t) from which the first section constitutes the S1 profile.

Those consecutive second and third stages allow avoiding undesired drug infusion spikes due to the presence at T2, in the tube 11, of residue of said combination of substances. Thus, the injection of glucose serum during the third stage and the complementarity of the profiles S1 and S2 allow extraction of the fluid such that the concentration of said combination of substances at the end E11 of the tube 11 changes according to the predetermined injection function ƒ(t).

In this example, the first injection profile is defined by the following equation:

$$S1 ( t ) = f ( t ) = {r}_{2} + {r}_{1} cos ( \frac{π ( t - T 1 )}{T} - \frac{π}{2} ) with {T}_{1} < t < {T}_{2} ,$$

where T is the duration of the second and the third stage, and r₁ and r₂ are predetermined coefficients. For example, r₁ and r₂ can be equal to 1.

In this example, the second injection profile is defined by the following equation:

$$S 2 ( t ) = f ( t ) = {r}_{2} + {r}_{1} cos ( \frac{π ( t - T 2 )}{T} - \frac{π}{2} ) with {T}_{2} < t < {T}_{e n d} ,$$

where T is the duration of the second and the third stage and T₂ is a predetermined offset time computed by Ω=V_{tube}+∫_{T}_{₁}^{T}^{₂}ƒ(t)dt, where V_{tube} the volume of the tube and Ω the total volume of drug solution to be delivered. S1 and S2—when placed end to end—define together the sinusoidal profile ƒ(t).

Although representative devices, components and methods have been described herein, those skilled in the art will recognize that various substitutions and modifications that may be made without departing from what is described and shown as well as defined by the appended claims.

1. A method of controlling the flow of a fluid in a tube (11) of an infusion pump (1), said fluid comprising at least one substance, this method comprising an estimation of the temporal evolution of the concentration of said at least one substance at an exit end (E11) of said tube (11) as a function of a size of said tube (11).

2. The method according to claim 1, in which said size is the length (L11) or the inner diameter or the inner volume of the tube (11).

3. The method according to claim 1 or 2, comprising injecting the fluid into the tube (11) according to a predetermined temporal injection profile (S1, S2, S3), said estimation being a function of this injection profile (S1, S2, S3).

4. The method according to claim 3, in which said temporal evolution of the concentration of the at least one substance along the tube (11) is estimated according to the following equation:
$$\frac{\partial U}{\partial t} = - V ( t ) \frac{\partial U}{\partial x} + D \frac{{\partial }^{2} U}{\partial {x}^{2}} + \partial ( x ) S ( t ) x ∈ [ 0 , L 11 ]$$
where U is the concentration of the at least one substance, V(t) is the velocity of the fluid flowing along the tube (11), D is a predetermined diffusion coefficient of the at least one substance, x is the coordinate along the tube (11) from its inlet end (111) where x=0 to its exit end (E11) where x=L11, L11 is the length of the tube (11), t is the time, ∂(x) is an indicator function which is equal to 1 at x=0 and 0 otherwise, and S(t) is the predetermined temporal injection profile.

5. The method according to claim 3 or 4, in which said injection profile (S3) is sinusoidal.

6. The method according to claim 3 or 4, comprising at least one injection cycle comprising in sequence:
a first stage in which a fluid is injected into the tube (11) so as to fill to its exit end (E11) with said fluid,
a second stage in which a fluid is injected into the tube (11) according to a first predetermined temporal injection profile (S1),
a third consecutive stage in which a fluid is injected into the tube (11) according to a second predetermined temporal injection profile (S2).

7. The method according to claim 6, in which the first injection profile (S1) is defined by the following equation: S1(t)=f(t) with T₁<t<T₂, T₁ being the time at which the second stage starts and T₂ the time at which the second stage ends, and where f(t) is a function, and in which the second injection profile (S2) is defined by the following equation: S2(t)=f(t) with T₂<t<T_{end}, T₂ being the time at which the third stage starts and T_{end} the time at which the third stage ends.

8. An infusion pump (1) having a tube (11) configured to flow a fluid therethrough and means adapted to carry out the method according to any of claims 1 to 7.

9. A computer program comprising instructions to cause the infusion pump (1) of claim 8 to carry out the method according to any of claims 1 to 7.

10. A computer-readable medium having stored thereon the computer program of claim 9.
